(19) United States
(12) Patent Application Publication
(10) Pub. No.: US 2025/0031959 A1
(43) Pub. Date: Jan. 30, 2025

(54) FUNDUS INSPECTION SYSTEM

(71) Applicant: Medimaging Integrated Solution, Inc., Hsinchu (TW)

(72) Inventors: Yung-En Kuo, Hsinchu (TW); Yu-Tsung Lee, Hsinchu (TW)

(21) Appl. No.: 18/784,232

(22) Filed: Jul. 25, 2024

(30) Foreign Application Priority Data
Jul. 27, 2023 (TW) 112128144

(51) Int. Cl. A61B 3/12; A61B 3/10; A61B 3/14

(52) U.S. Cl. CPC A61B 3/12; A61B 3/102; A61B 3/14

(57) ABSTRACT

A fundus inspection system includes an objective lens, a fundus imaging device, an optical coherence tomography device, a movable mirror, and a focus adjustment lens. The fundus imaging device captures the fundus image of an eyeball fundus along the optical axis of the objective lens and an imaging path. The optical coherence tomography device scans the eyeball fundus along the optical axis and a scanning path to capture the tomographic image of the fundus. An included angle is between the imaging path and the scanning path. The movable mirror, disposed at the intersection of the imaging path and the scanning path, moves to or away from the optical axis to optically couple the imaging path or the scanning path to the optical axis. The focus adjustment lens respectively or simultaneously captures the fundus image and the tomographic image corresponding to an eyeball state by moving the movable mirror.

### BACKGROUND OF THE INVENTION

### Field of the Invention

The present invention relates to a fundus inspection system, particularly to a fundus inspection system that combines a fundus imaging device with an optical coherence tomography device.

### Description of the Related Art

Fundus imaging devices (e.g., fundus cameras) are inspection tools used to observe the fundus of the eyeball, such as capturing images of the fundus surface to determine whether there are abnormalities in the retina, optic disc, blood vessel distribution, etc. Optical Coherence Tomography (OCT) is another optical imaging technology. It reflects two light rays through a reference arm and a sample arm respectively and then the light rays reach a light detector. Interference occurs in the light detector to form a tomographic image. Tomography images can be used to observe the sectional structure of the fundus.

In order to combine the fundus imaging device and the optical coherence tomography device in the same fundus inspection system, the conventional method is to use a dichroic beam splitter to combine the optical paths of the fundus imaging device and the optical coherence tomography device to observe the fundus of the eyes. The dichroic beam splitter is implemented with a multi-layer film that causes incident light of different wavelengths to produce different degrees of penetration or reflection. For example, incident light with a wavelength of greater than 900 nm has a higher transmittance (for example, more than 95%). Incident light with a wavelength of less than 900 nm has a high reflectivity (for example, more than 95%).

However, in order to satisfy the requirements of the fundus imaging device and the optical coherence tomography device, it is necessary to increase the number of coating layers on the surface of the dichroic beam splitter. This not only increases the difficulty and cost of manufacturing the dichroic beam splitter, but also generates multiple interference signals, which will affect the performance of the interference signals of the optical coherence tomography device and the imaging quality of the tomography image.

In summary, how to combine the fundus imaging device and the optical coherence tomography device in the same fundus inspection system and achieve better imaging quality is currently a goal that requires great efforts.

### SUMMARY OF THE INVENTION

The present invention provides a fundus inspection system, which moves a reflecting movable mirror to or away from an optical axis to optically couple the imaging path of a fundus imaging device or the scanning path of an optical coherence tomography device to the optical axis, thereby obtaining tomographic images with better qualities.

In an embodiment of the present invention, a fundus inspection system includes an objective lens, a fundus imaging device, an optical coherence tomography device, a movable mirror, and a focus adjustment lens. The objective lens has an optical axis. The fundus imaging device is configured to capture the fundus image of the fundus of an eyeball along the optical axis and an imaging path. The optical coherence tomography device is configured to scan the fundus of the eyeball along the optical axis and a scanning path to capture the tomographic image of the fundus. Therein there is an included angle between the imaging path and the scanning path. The movable mirror, disposed at the intersection of the imaging path and the scanning path, moves to or away from the optical axis to optically couple the imaging path or the scanning path to the optical axis. The focus adjustment lens, disposed between the objective lens and the movable mirror, moves to a location corresponding to the eyeball state of the eyeball along the optical axis based on the eyeball state. The focus adjustment lens is configured to respectively or simultaneously capture the fundus image and the tomographic image corresponding to the eyeball state by moving the movable mirror. The fundus imaging device and the optical coherence tomography device share the focus adjustment lens and respectively capture the fundus image generated by the fundus imaging device and the tomographic image generated by the optical coherence tomography device when the focus adjustment lens remains stationary.

Below, the embodiments are described in detail in cooperation with the drawings to make easily understood the technical contents, characteristics and accomplishments of the present invention.

### BRIEF DESCRIPTION OF THE DRAWINGS

FIG. 1A is a schematic diagram illustrating a fundus inspection system according to a first embodiment of the present invention;

FIG. 1B is a schematic diagram illustrating the partial optical path of a fundus inspection system according to a first embodiment of the present invention;

FIG. 2a and FIG. 2b are schematic diagrams illustrating a movable mirror according to a first embodiment of the present invention;

FIG. 3a and FIG. 3b are schematic diagrams illustrating a movable mirror according to a second embodiment of the present invention;

FIG. 4a and FIG. 4b are schematic diagrams illustrating a movable mirror according to a third embodiment of the present invention;

FIG. 5 is a schematic diagram illustrating a movable mirror according to a fifth embodiment of the present invention;

FIG. 6 is a schematic diagram illustrating a fundus inspection system according to a second embodiment of the present invention;

FIG. 7 is a schematic diagram illustrating a fundus inspection system according to a third embodiment of the present invention;

FIG. 8 is a schematic diagram illustrating a fundus inspection system according to a fourth embodiment of the present invention;

FIG. 9a is a schematic diagram illustrating the tomographic image of the fundus of a first participant captured by a conventional fundus inspection system;

FIG. 9b is a schematic diagram illustrating the tomographic image of the fundus of a first participant captured by a fundus inspection system according to an embodiment of the present invention;

FIG. 10a is a schematic diagram illustrating the tomographic image of the fundus of a second participant captured by a conventional fundus inspection system; and

FIG. 10b is a schematic diagram illustrating the tomographic image of the fundus of a second participant captured by a fundus inspection system according to an embodiment of the present invention.

### DETAILED DESCRIPTION OF THE INVENTION

Reference will now be made in detail to embodiments illustrated in the accompanying drawings. Wherever possible, the same reference numbers are used in the drawings and the description to refer to the same or like parts. In the drawings, the shape and thickness may be exaggerated for clarity and convenience. This description will be directed in particular to elements forming part of, or cooperating more directly with, methods and apparatus in accordance with the present disclosure. It is to be understood that elements not specifically shown or described may take various forms well known to those skilled in the art. Many alternatives and modifications will be apparent to those skilled in the art, once informed by the present disclosure.

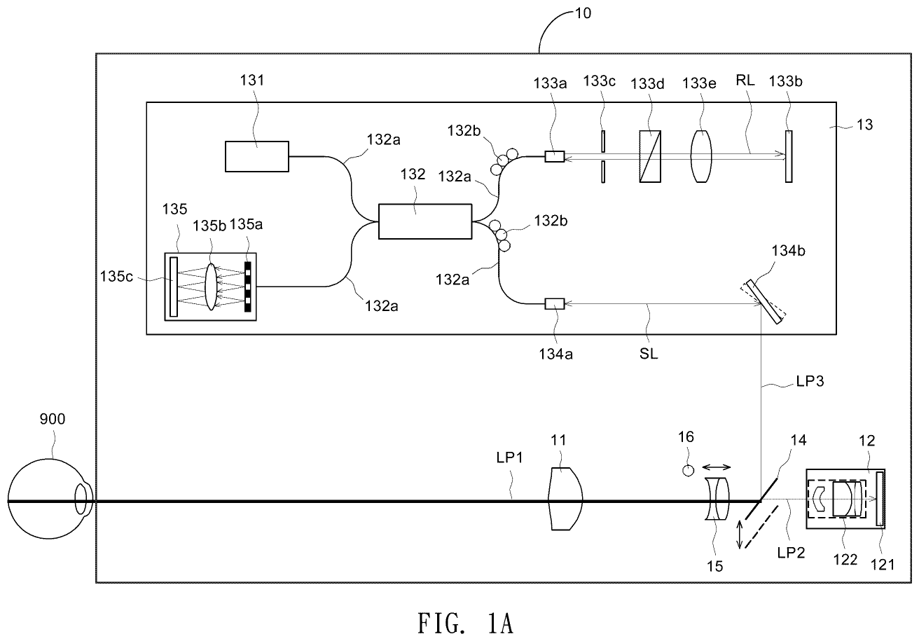

Referring to FIG. 1A, a fundus inspection system 10 of the present invention includes an objective lens 11, a fundus imaging device 12, an optical coherence tomography device 13, and a movable mirror 14. The objective lens 11 has an optical axis LP1 (i.e., a first optical path) that faces an eyeball 900. The fundus imaging device 12 captures the fundus image of the fundus of the eyeball 900 along the optical axis LP1 and an imaging path LP2. In the embodiment of FIG. 1A, the imaging path LP2 is the extension of the optical axis LP1. The optical coherence tomography device 13 scans the fundus of the eyeball 900 along the optical axis LP1 and a scanning path LP3 to capture a tomographic image of the fundus. In an embodiment, there is an included angle between the imaging path LP2 and the scanning path LP3. In the embodiment of FIG. 1A, the imaging path LP2 and the scanning path LP3 are perpendicular to each other. The movable mirror 14, disposed at the intersection of the imaging path LP2 and the scanning path LP3, moves to or away from the optical axis LP1 to optically couple the imaging path LP2 or the scanning path LP3 to the optical axis LP1.

The fundus inspection system 10 further includes a focus adjustment lens 15 that is disposed between the objective lens 11 and the movable mirror 14. The focus adjustment lens 15 may move to a location corresponding to the eyeball state of the eyeball 900 along the optical axis LP1 based on the eyeball state. The focus adjustment lens 15 respectively or simultaneously captures the fundus image and the tomographic image corresponding to the eyeball state by moving the movable mirror 14. In other embodiments, the focus adjustment lens 15 can also adjust the focal length by changing its curvature. For example, the focus adjustment lens 15 is a liquid crystal lens whose focal length is electronically controlled to achieve the optical zoom required by the fundus imaging device 12 and the optical coherence tomography device 13. When the focus adjustment lens 15 remains stationary, the fundus imaging device 12 and the optical coherence tomography device 13 share the focus adjustment lens 15 and respectively capture the fundus image generated by the fundus imaging device 12 and the tomographic image generated by the optical coherence tomography device 13. Based on the structure, the fundus imaging device 12 and the optical coherence tomography device 13 can share the focus adjustment lens, which can shorten the working time of adjusting the focal length and reduce the volume of the fundus inspection system.

Continuing from the foregoing description and referring to FIG. 1B, the partial optical path of the fundus inspection system of the present invention is introduced as follows. For example, different users have different vision or eye symptoms. Thus, each person's eyeball state is also different. For example, vision can be roughly divided into: normal vision, hyperopia, and myopia. (A) in FIG. 1B represents that the user's eyeball state is hyperopia, (B) in FIG. 1B represents that the user's eyeball state is normal, and (C) in FIG. 1B represents that the user's eyeball state is myopia. (A) in FIG. 1B represents that user A's eyeball has symptoms of hyperopia. Thus, the focus adjustment lens 15 must be moved to position X1 so that there is a distance D1 between the focus adjustment lens 15 and the objective lens 11. In (B) of FIG. 1B, if the eyeball of user B is normal, the focus adjustment lens 15 must be moved to position X2 so that there is a distance D2 between the focus adjustment lens 15 and the objective lens 11. In (c) of FIG. 1B, if the user C's eyeball is myopic, the focus adjustment lens 15 must be moved to position X3 so that there is a distance D3 between the focus adjustment lens 15 and the objective lens 11. Wherein, the position of the objective lens 11 is fixed. The focus adjustment lens 15 adjusts its position to position X1, position X2, or position X3 based on the eyeball state. Position X3 is closest to the objective lens 11 and position X1 is the farthest from the objective lens 11. Moreover, according to the distance between the focus adjustment lens 15 and the objective lens 11, the distance D1, the distance D2, and the distance D3 meet the following conditions: distance D1>distance D2>distance D3.

Please refer to FIG. 1A and FIG. 1B. In the usage state of user A, user A only needs to move the focus adjustment lens 15 to position X1 and move the movable mirror 14 to the optical axis LP1 such that the optical coherence tomography device 13 generates the tomographic image inspected by user A accordingly. Finally, user A moves the movable mirror 14 away from the optical axis LP1 such that the fundus imaging device 12 can generate the fundus image accordingly. In other words, without moving the focus adjustment lens 15 shared by the fundus imaging device 12 and the optical coherence tomography device 13, it is only necessary to move the movable mirror 14 such that the fundus imaging device 12 and the optical coherence tomography device 13 generate corresponding inspection images respectively.

As mentioned above, in the usage state of user B, user B moves the focus adjustment lens 15 shared by the fundus imaging device 12 and the optical coherence tomography device 13 to position X2 and moves the movable mirror 14 to the optical axis LP1, such that the optical coherence tomography device 13 generates the tomographic image. Finally, user B moves the movable mirror 14 away from the optical axis LP1, such that the fundus imaging device 12 can generate the fundus image. In the usage state, user C only needs to move the focus adjustment lens 15 to position X3 and then move the movable mirror 14 to the optical axis LP1, such that the optical coherence tomography device 13 generates the tomographic image. Finally, user C moves the movable mirror 14 away from the optical axis LP1, such that the fundus imaging device 12 can generate the fundus image.

In the conventional inspection process, adjusting the focal length has always been a professional operation that consumes time and requires skill. Users of the conventional technology must adjust the focal lengths of the fundus photography device and the optical tomography device separately. However, because the optical designs of the two are completely different, the conventional technology must firstly adjust the focal length of the fundus photography device once. After the fundus photography device captures images, the focal length of the optical tomography device is adjusted. Finally, tomographic images are captured. Therefore, the conventional technology not only increases the cost of components, but also separately adjusts the focal lengths. Thus, the number of lens groups cannot be reduced and the overall size cannot be reduced. Besides, the operation is also complicated for the user. It must be noted that, in the present invention, it is only necessary to adjust the position of the focal length of the focus adjustment lens 15 to a target position once. In subsequent operations, the movable mirror 14 can be used to control whether to capture tomographic images and fundus images simultaneously or separately. Therefore, the fundus imaging device 12 and the optical coherence tomography device 13 of the present invention can share the focus adjustment lens 15, thereby shortening the focusing response time, reducing costs, and reducing the volume of the fundus inspection system 10.

Please refer to FIG. 1A. The fundus imaging device includes a light-emitting component 16, which is arranged away from the optical axis LP1. In one embodiment, the illumination light generated by the point-shaped light-emitting component 16 is directly irradiated to the objective lens 11, so that components such as relay lenses and reflectors in the illumination system can be omitted, thereby reducing the volume of the fundus inspection system. In one embodiment, a distance from the light-emitting component 16 to the objective lens 11 is less than or equal to a distance from the focus adjustment lens 15 to the objective lens 11.

The fundus imaging device 12 includes an image sensor 121 and an imaging lens 122. The imaging lens 122 is disposed on the incident side of the image sensor 121 to converge the reflected light from the fundus of the eyeball 900 and image it on the image sensor 121. The image sensor 121 can receive the reflected light from the fundus to form the corresponding fundus image.

The optical coherence tomography device includes a scanning light source 131, a coupler 132, a reference path RL, a sampling path SL, and a spectrometer 135. The scanning light source 131 is configured to generate scanning light. For example, the scanning light source 131 may be a superluminescent diode (SLD). The coupler 132 is optically coupled to the scanning light source 131 such that the scanning light is divided into reference light and sampling light. The coupler 132 respectively guides the reference light and the sampling light to the reference path RL and the sampling path SL to output them. For example, the coupler 132 optically couples the scanning light source 131 with an optical fiber 132a and outputs the reference light and the sampling light to the reference path RL and the sampling path SL respectively with the optical fiber 132a.

The reference path RL at least includes a collimator 133a and a reference reflector 133b. The collimator 133a is disposed at one end of the optical fiber 132a, so that the reference light is output from the collimator 133a. The reference light travels to the reference reflector 133b along the reference path and then the reference reflector 133b reflects the reference light back to the collimator 133a. It can be understood that the reference path RL may include other appropriate optical components. For example, an aperture 133c, a dispersion compensator (DC) 133d, and a lens 133e can be disposed in the reference path RL to stabilize the quality of the reference light. The detailed structure of the reference path RL is well known to those with ordinary skills in the art of the present invention, so it will not be reiterated.

The sampling path SL at least includes a collimator 134a and a scanning reflector 134b. The sampling light output from the collimator 134a travels to the scanning mirror 134b along the sampling path SL and then sequentially passes through the movable mirror 14 and the objective lens 11 to illuminate the fundus of the eyeball 900. The sampling light reflected from the fundus returns to the coupler 132 along the original path. The scanning reflector 134b can deflect the path of the sampling light in a rotation manner or other methods, thereby changing different positions irradiated by the sampling light on the fundus of the eyeball 900. It can be understood that the specific area of the fundus of the eyeball 900 can be completely scanned by sequentially changing the position where the sampling light is irradiated to the fundus. In one embodiment, the scanning reflector 134b can be implemented with microelectromechanical systems (MEMS). In one embodiment, the optical coherence tomography device 13 may include a polarization controller 132b, which is optically coupled to the coupler 132 to polarize the scanning light that travels to the reference path RL and the sampling path SL.

The spectrometer 135 is optically coupled to the coupler 132. For example, the spectrometer 135 is optically coupled to the coupler 132 via an optical fiber 132a. The spectrometer 135 is used to receive the optical signals returned from the reference path RL and the sampling path SL. In one embodiment, the spectrometer 135 includes a diffraction grating 135a, a lens 135b and a line scan camera 135c to detect the light signals caused by interfering with the reference light and the sampling light returned from the reference path RL and the sampling path SL and to generate corresponding tomographic images.

Continuing from the foregoing description, the movable mirror 14 is disposed at the intersection of the imaging path LP2 and the scanning path LP3. The movable mirror 14 can move to or away from the optical axis LP1 to couple the imaging path LP2 or the scanning path LP3 to the optical axis LP1. For example, in the embodiment shown in FIG. 1A, when the movable mirror 14 moves to the optical axis LP1, the scanning path LP3 is optically coupled to the optical axis LP1. At this time, the fundus imaging device 12 cannot receive the reflected light from the fundus of the eyeball 900. When the movable mirror 14 moves away from the optical axis LP1, the imaging path LP2 is optically coupled to the optical axis LP1. At this time, the optical coherence tomography device 13 cannot receive the reflected light from the fundus of the eyeball 900.

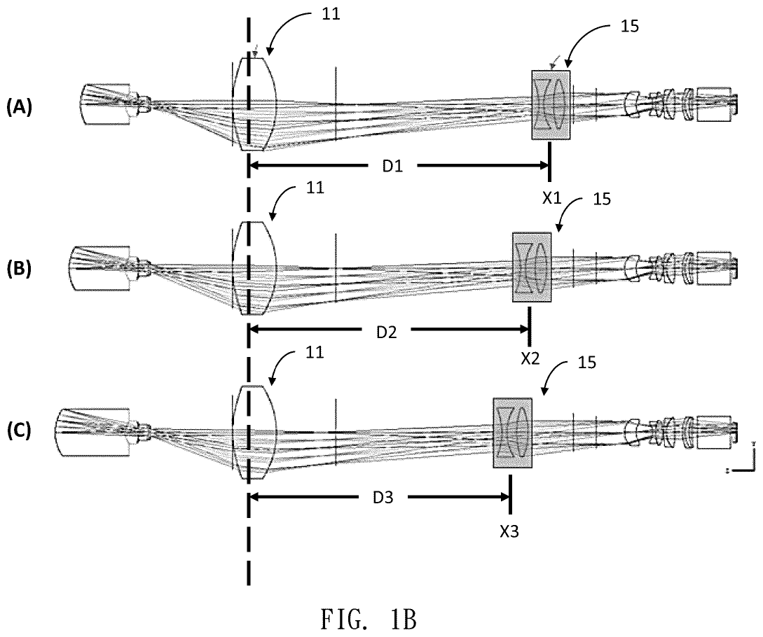

Please refer to FIG. 2a and FIG. 2b. In the embodiment, the movable mirror 14 includes a reflector 142c and a first pivot 141. The reflector 142c has a fixed end c1 and a movable end c2. The first pivot 141 is provided at the fixed end c1 and the movable end c2 rotates about the first pivot 141, so that the reflector 142c can rotate to move to or away from the optical axis LP1. In addition, the movable mirror 14 may also include a driving controller that controls the reflector 142c to rotate about the first pivot 141 and that controls the moving distance and position of the movable end c2 of the reflector 142c.

As illustrated in FIG. 2a, in State S11, the reflector 142c rotates about the first pivot 141 to move the movable mirror 14 to the optical axis LP1.

When the movable end c2 is located at the position A1, the scanning path LP3 is optically coupled to the optical axis LP1 such that the tomographic images of the fundus can be captured.

As illustrated in FIG. 2b, in State S12, the reflector 142c rotates about the first pivot 141. When the movable end c2 moves from position A1 to position A2, the movable mirror 14 moves away from the optical axis LP1. At this time, the imaging path LP2 is optically coupled to the optical axis LP1 such that the fundus image of the fundus can be captured.

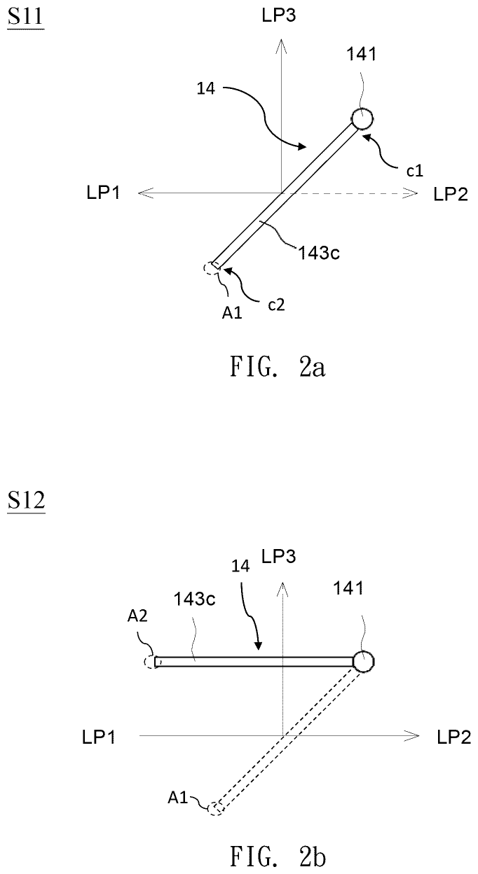

Please refer to FIG. 3a and FIG. 3b. In this embodiment, the movable mirror 14 includes a reflector 142c and a second pivot 141′. The reflector 142c has a first surface s1 and a second surface s2 opposite each other. The second pivot 141′ is disposed on one side of the first surface s1 and connected to the reflector 142c. The reflector 142c rotates about the second pivot 141′, so that the reflector 142c moves to or away from the optical axis. In addition, the movable mirror 14 may also include a driving controller that controls the reflector 142c to rotate about the second pivot 141′ and that controls the rotation angle and position of the reflector 142c.

As illustrated in FIG. 3a, in State S21, the reflector 142c rotates about the second pivot 141′ and the second surface s2 of the reflector 142c faces the optical axis LP1. When the reflector 142c is located at the position A3, the movable mirror 14 has moved to the optical axis LP1. At this time, the scanning path LP3 is optically coupled to the optical axis LP1, such that the tomographic image of the fundus can be captured.

As illustrated in FIG. 3b, in State S22, the reflector 142c rotates about the second pivot 141′. When the reflector 142c moves from position A3 to position A4, the second surface s2 of the reflector 142c moves away from the optical axis. LP1. At this time, the imaging path LP2 is optically coupled to the optical axis LP1 and the fundus image of the fundus can be captured.

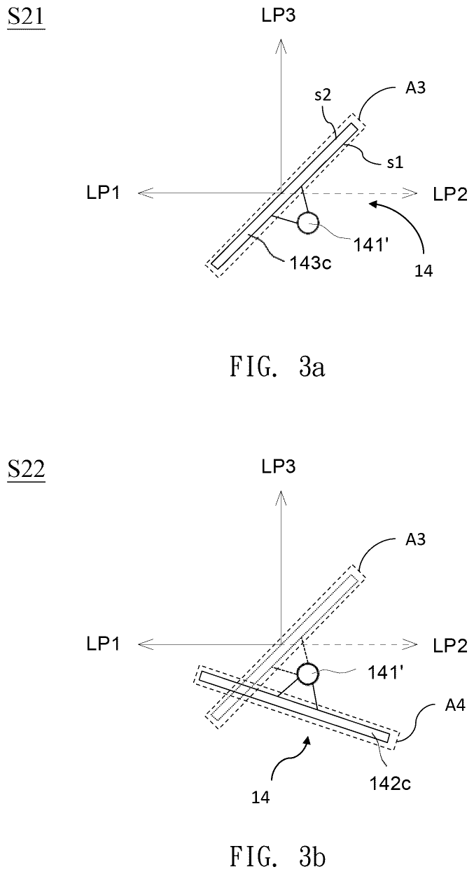

Please refer to FIG. 4a and FIG. 4b. The movable mirror 14 includes a bracket 142 and a reflector 142c. The bracket 142 is provided with a placement area 142a and a perspective channel 142b adjacent thereto. The perspective channel 142b is openwork and located along the optical axis LP1. The reflector 142c, located in the bracket 142, moves between the placement area 142a and the perspective channel 142b. In addition, the movable mirror 14 may also include a driving controller that controls the reflector 142c to move between the placement area 142a and the perspective channel 142b and that controls the moving path and direction of the reflector 142c.

As illustrated in FIG. 4a, in State S31, the reflector 142c is far away from the optical axis LP1 when the reflector 142c moves from position A5 to the placement area 142a. At this time, the imaging path LP2 is optically coupled to the optical axis LP1, such that the fundus images of the fundus are captured.

As illustrated in FIG. 4b, in State S32, the reflector 142c blocks the perspective channel 142b, which means that the reflector 142c has moved to the optical axis LP1 when the reflector 142c is located at position A5. At this time, the scanning path LP3 is optically coupled to the optical axis LP1, the tomographic images of the fundus can be captured.

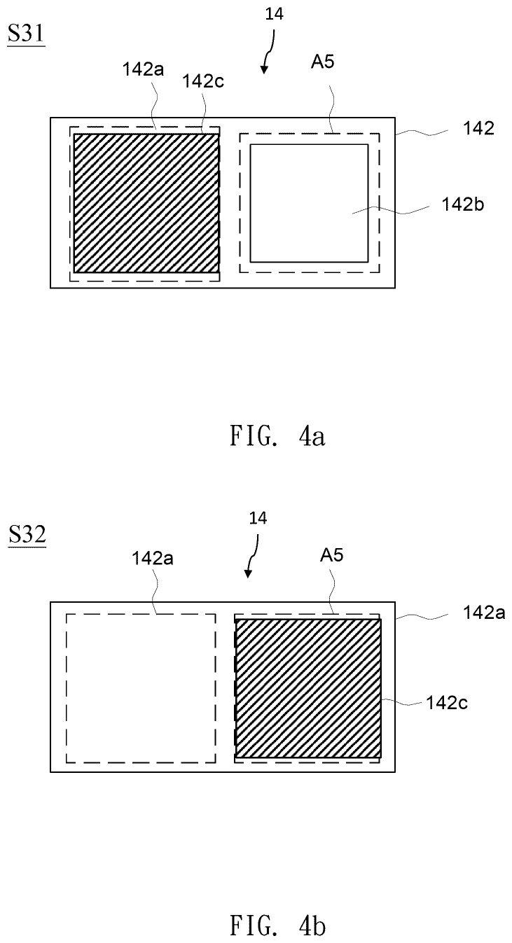

Please refer to FIG. 5. The movable mirror 14 includes a bracket 142, a reflector 142c, a magnetic coil 142d, and a magnetic component 142e. The structural features of the bracket 142 and the reflector 142c are the same as those in FIGS. 4a and 4b, so they will not be reiterated. The magnetic coil 142d is disposed on the bracket 142 and is close to the placement area 142a. The magnetic component 142e is disposed on the reflector 142c and disposed adjacent to the magnetic coil 142d. The magnetic coil 142d receives a current to generate a magnetic force for attracting or repelling the magnetic component 142e, thereby moving the magnetic component 142e.

In the state where the magnetic force of the magnetic coil 142d repels the magnetic component 142e, the reflector 142c is located in the perspective channel 142b, which means that the reflector 142c has moved to the optical axis LP1. At this time, the scanning path LP3 is optically coupled to the optical axis LP1, such that the tomographic images of fundus can be captured.

In the state where the magnetic force of the magnetic coil 142d attracts the magnetic component 142e, the reflector 142c is located in the placement area 142a, which means that the reflector 142c is far away from the optical axis LP1. At this time, the imaging path LP2 is coupled to the optical axis LP1, such that the fundus images of the fundus can be captured.

According to the foregoing description, regardless of whether the movable mirror rotates or displaces, the speed of the movable mirror 14 moving to or away from the optical axis LP1 can reach millisecond level, which can improve the shooting efficiency when the movable mirror 14 rapidly switches the optical path. The operator can also use the fundus inspection system 10 of the present invention to obtain the tomographic images and fundus images of the fundus of the eyeball 900 separately or to obtain tomographic images and fundus images simultaneously. For example, in the embodiment of FIG. 1A, when the operator wants to obtain tomography images alone, the movable mirror 14 first moves away from the optical axis LP1 to optically couple the imaging path LP2 to the optical axis LP1. At this time, the operator can inspect the fundus image of the eyeball 900 and thereby confirm the tomographic range of the fundus. After confirming the tomographic range, the movable mirror 14 moves to the optical axis LP1 to optically couple the scanning path LP3 to the optical axis LP1. At this time, the fundus inspection system 10 of the present invention can perform tomography and obtain the tomographic image of the fundus of the eyeball 900. It can be understood that the movable mirror 14 only needs to move away from the optical axis LP1 to optically couple the imaging path LP2 to the optical axis LP1 when the operator wants to obtain the fundus image alone. The operator can inspect and capture the fundus image of the eyeball 900. In addition, as mentioned above, when the optical path is quickly switched by the movable mirror 14, fundus images and tomographic images can be obtained simultaneously. Alternatively, in one embodiment, the fundus inspection system 10 of the present invention can firstly obtain the tomographic image of the fundus of the eyeball 900 and then capture the fundus image of the fundus of the eyeball 900.

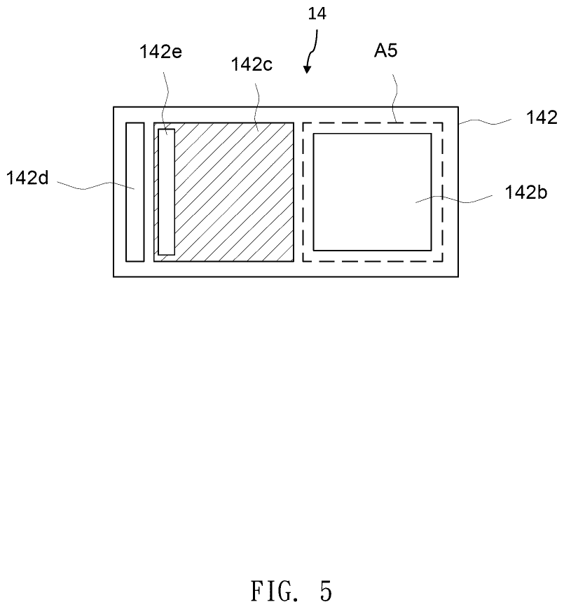

Please refer to FIG. 6. In another embodiment, the fundus inspection system of the present invention further includes a fixed eye lamp device 17 and a beam splitter 18. The fixed eye lamp device 17 can generate a fixed eye image and output the fixed eye image along a fixed eye path LP4. For example, the fixed eye light device 17 may include a display panel and either a relay lens or a focus adjustment lens. The beam splitter 18 optically couples the fixed eye path LP4 to the optical axis LP1, so that the fixed eye image can be formed on the fundus of the eyeball 900. In the embodiment of FIG. 6, the beam splitter 18 is disposed on the optical axis LP1 between the objective lens 11 and the movable mirror 14, but the present invention is not limited thereto.

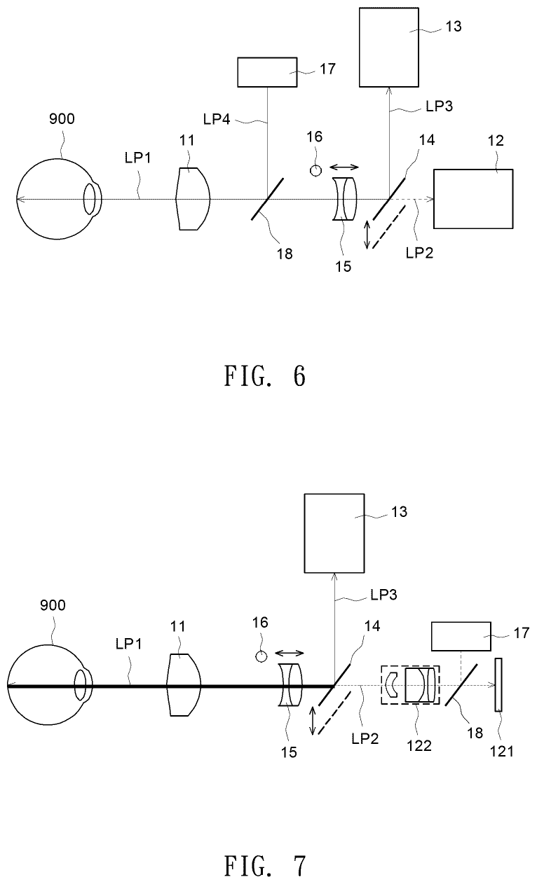

Please refer to FIG. 7. In one embodiment, the beam splitter 18 can be disposed on the imaging path LP2. In the embodiment of FIG. 7, the beam splitter 18 is disposed between the movable mirror 14 and the image sensor 121 of the fundus imaging device 12. Preferably, the fixed eye lamp device 17 and the image sensor 121 of the fundus imaging device 12 are located on an equivalent focal plane. Based on the structure, when the image sensor 121 is at the focus, the fixed eye lamp device 17 is also at the focus, which can shorten the time for adjusting the focal distance.

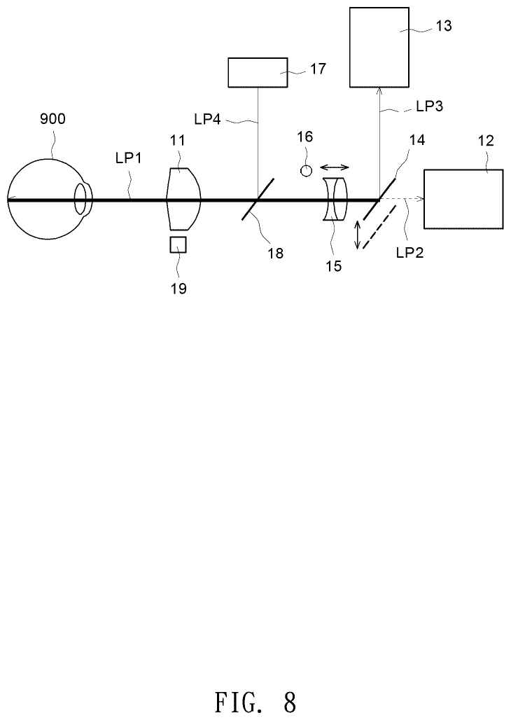

Please refer to FIG. 8. In another embodiment, the fundus inspection system of the present invention further includes an eyeball tracking module 19. The eyeball tracking module 19 can capture the external eye image of the eyeball 900 to locate the pupil position of the eyeball 900. For example, the eyeball tracking module 19 can generate the pupil center coordinates of the eyeball 900 to facilitate the automatic adjustment of the relative positions of the fundus inspection system of the present invention and the eyeball 900, so that the fundus inspection system of the present invention faces the eyeball 900. Alternatively, the fundus inspection system of the present invention outputs corresponding prompt messages based on the pupil center coordinates to guide the operator to adjust the relative positions of the fundus inspection system of the present invention and the eyeball 900, so that the fundus inspection system of the present invention faces the eyeball 900.

Based on the foregoing structure, the fundus inspection system 10 of the present invention employs a reflective movable mirror 14 to switch the optical path. Therefore, the tomographic scan images obtained by the fundus inspection system 10 of the present invention will not suffer from image quality degradation due to the multi-layer coating design of the dichroic beam splitter.

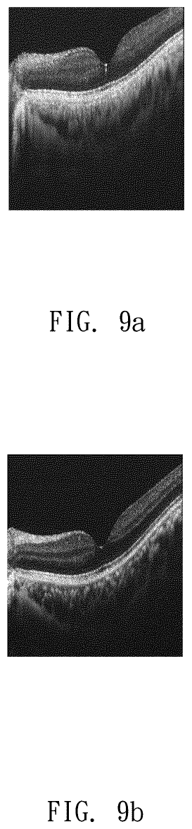

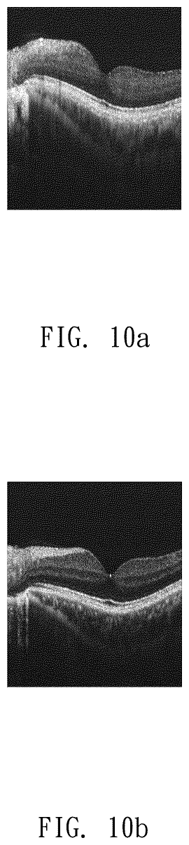

For example, please refer to FIGS. 9a-10b. FIG. 9a illustrates a tomographic image obtained by scanning the fundus of a first participant using a conventional fundus inspection system (using a dichroic beam splitter). FIG. 9b illustrates a tomographic image obtained by scanning the fundus of the first participant using the fundus inspection system of the present invention. FIG. 10a illustrates a tomographic image obtained by scanning the fundus of a second participant using a conventional fundus inspection system (using a dichroic beam splitter). FIG. 10b illustrates a tomographic image obtained by scanning the fundus of the second participant using the fundus inspection system of the present invention. It can be seen from FIG. 9a and FIG. 10a that the tomographic image output by the conventional fundus inspection system has overlapping images (such as white bright spots) and the image is relatively blurry. In contrast, the tomographic image output by the fundus inspection system of the present invention is relatively clear and the human body of the tomographic image is clear. After calculating the signal to noise ratio (SNR), the tomographic image output by the fundus inspection system of the present invention also has a higher SNR, where the SNR of the tomographic image in FIG. 9a is 12.5, the signal-to-noise ratio of the tomographic image in FIG. 9b is 14.0, the signal-to-noise ratio of the tomographic image in FIG. 10a is 13.6, and the signal-to-noise ratio of the tomographic image in FIG. 10b is 15.6.

In conclusion, the fundus inspection system of the present invention uses a reflective movable mirror to selectively switch the imaging path of the fundus imaging device or the scanning path of the optical coherence tomography device for fundus inspection. Therefore, compared with the multi-layer coating design of a dichroic beam splitter, the tomographic scan images obtained by the fundus inspection system of the present invention have better image quality.

The embodiments described above are only to exemplify the present invention but not to limit the scope of the present invention. Therefore, any equivalent modification or variation according to the shapes, structures, features, or spirit disclosed by the present invention is to be also included within the scope of the present invention.

What is claimed is:

1. A fundus inspection system comprising:
an objective lens having an optical axis;
a fundus imaging device configured to capture a fundus image of a fundus of an eyeball along the optical axis and an imaging path;
an optical coherence tomography device configured to scan the fundus of the eyeball along the optical axis and a scanning path to capture a tomographic image of the fundus, wherein there is an included angle between the imaging path and the scanning path;
a movable mirror, disposed at an intersection of the imaging path and the scanning path, moving to or away from the optical axis to optically couple the imaging path or the scanning path to the optical axis; and
a focus adjustment lens, disposed between the objective lens and the movable mirror, moving to a location corresponding to an eyeball state of the eyeball along the optical axis based on the eyeball state, the focus adjustment lens is configured to respectively or simultaneously capture the fundus image and the tomographic image corresponding to the eyeball state by moving the movable mirror;
wherein when the focus adjustment lens remains stationary, the fundus imaging device and the optical coherence tomography device share the focus adjustment lens and respectively capture the fundus image generated by the fundus imaging device and the tomographic image generated by the optical coherence tomography device.

2. The fundus inspection system according to claim 1, wherein the imaging path and the scanning path are perpendicular to each other.

3. The fundus inspection system according to claim 1, wherein the fundus imaging device includes a light-emitting component disposed away from the optical axis and configured to directly emit illumination light to the objective lens.

4. The fundus inspection system according to claim 1, wherein the fundus imaging device includes:
an image sensor configured to receive reflected light from the fundus of the eyeball to form the fundus image; and
an imaging lens disposed on an incident side of the image sensor to converge the reflected light to form an image on the image sensor.

5. The fundus inspection system according to claim 1, wherein the optical coherence tomography device includes:
a scanning light source configured to generate scanning light;
a coupler optically coupled to the scanning light source and configured to divide the scanning light into reference light and sampling light, wherein the reference light travels along a reference path and the sampling light travels along a sampling path;
a reference reflector disposed in the reference path and configured to reflect the reference light to the coupler along the reference path;
a scanning reflector disposed in the sampling path and configured to deflect the sampling light to scan the fundus of the eyeball along the scanning path, wherein the sampling light reflected by the fundus travels back to the coupler along the optical axis, the scanning path, and the sampling path; and
a spectrometer optically coupled to the coupler and configured to receive the reference light and the sampling light and generate the tomographic image corresponding thereto.

6. The fundus inspection system according to claim 1, wherein the movable mirror includes:
a reflector having a fixed end and a movable end opposite to each other; and
a first pivot disposed at the fixed end, and the movable end is configured to rotate about the first pivot to move to or away from the optical axis.

7. The fundus inspection system according to claim 1, wherein the movable mirror includes:
a reflector having a first surface and a second surface opposite to each other; and
a second pivot disposed on a side of the first surface, connected with the reflector, and the reflector is configured to rotate about the second pivot to move to or away from the optical axis.

8. The fundus inspection system according to claim 1, wherein the movable mirror includes:
a bracket provided with a placement area and a perspective channel adjacent thereto, and the perspective channel is openwork and located along the optical axis; and
a reflector located in the bracket and configured move between the placement area and the perspective channel, wherein the reflector blocks the perspective channel when the reflector is disposed in the perspective channel, and the optical axis passes through the perspective channel when the reflector is disposed in the placement area.

9. The fundus inspection system according to claim 8, wherein the movable mirror includes:
a magnetic coil disposed on the bracket and close to the placement area; and
a magnetic component disposed on the reflector and disposed adjacent to the magnetic coil;
wherein the magnetic coil is configured to receive a current to generate a magnetic force for attracting or repelling the magnetic component, thereby moving the magnetic component, the reflector is disposed in the perspective channel when the magnetic force generated by the magnetic coil repels the magnetic component, and the reflector is disposed in the placement area when the magnetic force generated by the magnetic coil attracts the magnetic component.

10. The fundus inspection system according to claim 1, further comprising:
a fixed eye lamp device configured to generate a fixed eye image and output the fixed eye image along a fixed eye path; and
a beam splitter disposed in the optical axis or the imaging path and configured to combine the fixed eye path with the optical axis to form the fixed eye image on the fundus of the eyeball.

11. The fundus inspection system according to claim 10, wherein the beam splitter is disposed between the objective lens and the movable mirror.

12. The fundus inspection system according to claim 10, wherein the beam splitter is disposed between the movable mirror and an image sensor of the fundus imaging device and the fixed eye lamp device and the image sensor of the fundus imaging device are disposed on a equivalent focal plane.

13. The fundus inspection system according to claim 1, wherein the fundus imaging device includes an eyeball tracking module configured to capture an external eye image of the eyeball to locate a pupil position of the eyeball.
